(19) United States
(12) Patent Application Publication
(10) Pub. No.: US 2005/0059837 A1
(43) Pub. Date: Mar. 17, 2005

(54) Method for producing butyl acrylate

(76) Inventors: Cynthia Kiest Cooper, Houston, TX (US); Rene Zamarripa, Houston, TX (US)

Correspondence Address: Kenneth Crimaldi;Rohm and Haas Company, 100 Independence Mall West, Philadelphia, PA, 19106, US

(21) Appl. No.: 10/862,500

(22) Filed: Jun. 7, 2004

Related U.S. Application Data
(60) Provisional application No. 60/503,449, filed on Sep. 15, 2003.

(51) Int. Cl. C07C 69/66; C07C 69/52; C07C 67/48

(52) U.S. Cl. 560/205

(57) ABSTRACT

A continuous process for producing n-butyl acrylate substantially free of acrylic acid and n-butyl acetate and for recovering acrylic acid, n-butyl acrylate, n-butanol and water from an esterification reactor mixture containing acrylic acid, n-butyl acrylate, n-butanol, water, heavy ends, and acid catalyst; wherein two process units are run in parallel, each comprising an esterification reactor and a dehydration distillation column; and wherein a first process unit has a hydrolysis reactor, a cracking reactor and an acrylic acid separation column and a second process unit has a bleed stripper, a recycle tank and a neutralization and acidification system; with interconnections between the two process units that are designed to maximize yield and purity of the products from both units.

This invention relates to a method for combining acrylic acid (AA) and n-butanol, and processing the reaction products to produce n-butyl acrylate (BA) having reduced levels of acrylic acid and n-butyl acetate in improved yield.

A process for producing n-butyl acrylate is disclosed in related U.S. Pat. Nos. 5,877,345; 5,990,343 and 6,180,819. This process has two new process components, one related to the recovery and recycle of valuable reactants from their higher boiling adducts via hydrolysis and cracking reactions, and a second component related to improved distillation of a crude product yielding BA substantially free of AA. The aforementioned patents are incorporated by reference herein.

The present invention relates to a method for running a process unit described in the previous patents together with a second process unit having a different configuration, with interconnections between the two process units that are designed to maximize yield and purity of the products from both units. The present invention also relates to a method for combining parts of the two units into a single process unit for improved yield and purity.

### STATEMENT OF THE INVENTION

The present invention is directed to a continuous process for producing n-butyl acrylate substantially free of acrylic acid and n-butyl acetate and for recovering acrylic acid, n-butyl acrylate, n-butanol and water from an esterification reactor mixture containing acrylic acid, n-butyl acrylate, n-butanol, water, heavy ends, and acid catalyst; wherein two process units are run in parallel, each comprising an esterification reactor and a dehydration distillation column; and wherein a first process unit has a hydrolysis reactor, a cracking reactor and an acrylic acid separation column and a second process unit has a bleed stripper, a recycle tank and a neutralization and acidification system; comprising steps of, with each step occurring in both the first and the second process unit unless otherwise indicated:
a) feeding to the esterification reactors acrylic acid and n-butanol, in a molar ratio of from 1 to 1.1 to 1 to 1.7, and the acid catalyst;
b) reacting the acrylic acid and n-butanol to yield n-butyl acrylate in a conversion of at least 60% on acrylic acid, and yielding the esterification reaction mixture comprising acrylic acid, n-butyl acrylate, n-butanol, di-n-butyl ether, n-butyl acetate, water, heavy ends, and acid catalyst;
c) withdrawing a reactor bleed stream from the continuously converting esterification reactor mixture while concurrently distilling acrylic acid, n-butyl acrylate, n-butanol and water from the esterification reaction mixture;
d) feeding the reactor bleed stream from the second process unit reactor to a bleed stripper unit from which a distillate comprising acrylic acid, n-butyl acrylate and n-butanol is removed;
e) feeding total aqueous and organic feed streams from each process unit comprising the reactor bleed stream from the first process unit and the bleed stripper bottom stream from the second process unit, water or reactor aqueous distillate from the first process unit, optionally a strong acid selected from a mineral acid or sulfonic acid, and optionally additional heavy ends, to the hydrolysis reactor in the first process unit, which is maintained at 90° to 140° C., 50 to 1000 mm Hg pressure, and a residence time of 0.5 to 20 hours based on the total aqueous and organic feed stream;
f) in the first process unit, distilling an overhead stream containing acrylic acid, n-butyl acrylate, n-butanol, and water from the hydrolysis reactor while maintaining a hydrolysis reactor liquid concentration of from 5 to 40 weight % water and at least 1 weight % acid, the acid comprising the acid catalyst and the optional strong acid; and condensing the overhead stream;
g) in the first process unit, separating from the condensed overhead stream from step f) an organic phase comprising n-butyl acrylate, n-butanol, and acrylic acid, and an aqueous phase comprising primarily water, and acrylic acid, and n-butanol; feeding the separated organic phase to the esterification reactor; and feeding the separated aqueous phase to the hydrolysis reactor;
h) in the first process unit, withdrawing from the hydrolysis reactor from 20 to 70 weight %, based on the total aqueous and organic feed stream, of a hydrolysis reactor bleed stream; and feeding up to 100% of the hydrolysis reactor bleed stream to a cracking reactor maintained at 90° to 140° C., a pressure of from 20 to 200 mm Hg, and a residence time of 0.5 to 20 hours based on the fed reactor bleed stream;
i) in the first process unit, distilling from the cracking reactor a cracking reactor overhead stream comprising acrylic acid, n-butyl acrylate, n-butanol, and water while maintaining a cracking reactor liquid concentration of at least 7.5 weight % acid; condensing the cracking reactor overhead stream; and recycling to the esterification reactor the condensed cracking reactor overhead stream comprising acrylic acid, n-butyl acrylate, n-butanol, and water;
j) distilling from the esterification reactor, concurrently with above steps c) through i), a vaporized mixture comprising acrylic acid, n-butyl acrylate, n-butanol, and water;
k) condensing the vaporized mixture to provide a first condensate comprising an organic phase and an aqueous phase; returning from 0 to 30 percent of the organic phase to an entrainment separator surmounting the esterification reactor;
l) in the first process unit, feeding from 70 to 100 percent of the organic phase and from 50 to 100 percent of the aqueous phase to an acrylic acid separation column; distilling from the acrylic acid separation column, at a pressure of from 35 to 800 mm Hg, in an aqueous mode and at an aqueous reflux ratio of 8.5:1 to 17:1, an overhead mixture comprising an azeotropic mixture of n-butanol, butyl acrylate and water; and removing from the distillation column an acrylic acid-rich bottom stream;
m) in the first process unit, recycling the acrylic acid-rich bottom stream from the acrylic acid separation column to the esterification reactor; condensing the overhead mixture to provide a second condensate; separating the second condensate into a n-butyl acrylate-rich organic phase and a separated aqueous phase; and removing the n-butyl acrylate-rich organic phase substantially free of acrylic acid;
n) in the second process unit, feeding from 70 to 100 percent of the organic phase from step k) to the neutralization and acidification system to produce an n-butyl acrylate-rich organic phase low in acrylic acid;
o) feeding an n-butyl acrylate-rich organic phase low in acrylic acid from step m) in the first process unit or step n) in the second process unit to the dehydration distillation columns, which produce overhead streams comprising butyl acetate, n-butanol and n-butyl acrylate, and bottom streams rich in n-butyl acrylate;
p) feeding a portion from 1 to 15% of the overhead stream comprising butyl acetate, n-butanol and n-butyl acrylate from step o) in the first process unit to the recycle tank in the second process unit, from which a recycle feed stream is sent to the second process unit esterification reactor.

The invention is further directed to a continuous process for producing n-butyl acrylate substantially free of acrylic acid and n-butyl acetate and for recovering acrylic acid, n-butyl acrylate, n-butanol and water from an esterification reactor mixture containing acrylic acid, n-butyl acrylate, n-butanol, water, heavy ends, and acid catalyst; comprising steps of:
a) feeding to an esterification reactor acrylic acid and n-butanol, in a molar ratio of from 1 to 1.1 to 1 to 1.7, and the acid catalyst;
b) reacting the acrylic acid and n-butanol to yield n-butyl acrylate in a conversion of at least 60% on acrylic acid, and yielding the esterification reaction mixture comprising acrylic acid, n-butyl acrylate, n-butanol, di-n-butyl ether, n-butyl acetate, water, heavy ends, and acid catalyst;
c) withdrawing a reactor bleed stream from the continuously converting esterification reactor mixture while concurrently distilling acrylic acid, n-butyl acrylate, n-butanol and water from the esterification reaction mixture;
d) feeding the reactor bleed stream to a bleed stripper unit from which a distillate comprising acrylic acid and n-butyl acrylate is removed;
e) feeding the bleed stripper bottom stream, water, optionally a strong acid selected from a mineral acid or sulfonic acid, and optionally additional heavy ends, to a hydrolysis reactor, which is maintained at 90° to 140° C., 50 to 1000 mm Hg pressure, and a residence time of 0.5 to 20 hours based on the total aqueous and organic feed stream;
f) distilling an overhead stream containing acrylic acid, n-butyl acrylate, n-butanol, and water from the hydrolysis reactor while maintaining a hydrolysis reactor liquid concentration of from 5 to 40 weight % water and at least 1 weight % acid, the acid comprising the acid catalyst and the optional strong acid; and condensing the overhead stream;
g) separating from the condensed overhead stream from step f) an organic phase comprising n-butyl acrylate, n-butanol, and acrylic acid, and an aqueous phase comprising primarily water, and acrylic acid, and n-butanol; feeding the separated organic phase to an esterification reactor recycle tank; and feeding the separated aqueous phase to the hydrolysis reactor;
h) withdrawing from the hydrolysis reactor from 20 to 70 weight %, based on the total aqueous and organic feed stream, of a hydrolysis reactor bleed stream; and feeding up to 100% of the hydrolysis reactor bleed stream to a cracking reactor maintained at 90° to 140° C., a pressure of from 20 to 200 mm Hg, and a residence time of 0.5 to 20 hours based on the fed reactor bleed stream;
i) distilling from the cracking reactor a cracking reactor overhead stream comprising acrylic acid, n-butyl acrylate, n-butanol, and water while maintaining a cracking reactor liquid concentration of at least 7.5 weight % acid; condensing the cracking reactor overhead stream; and recycling to the esterification reactor recycle tank the condensed cracking reactor overhead stream comprising acrylic acid, n-butyl acrylate, n-butanol, and water;
j) distilling from the esterification reactor, concurrently with above steps c) through i), a vaporized mixture comprising acrylic acid, n-butyl acrylate, n-butanol, and water;
k) condensing the vaporized mixture to provide a first condensate comprising an organic phase and an aqueous phase; returning from 0 to 30 percent of the organic phase to an entrainment separator surmounting the esterification reactor;
l) feeding from 70 to 100 percent of the organic phase from step k) to the neutralization and acidification system to produce an n-butyl acrylate-rich organic phase low in acrylic acid;
m) feeding an n-butyl acrylate-rich organic phase low in acrylic acid from step l) to a dehydration distillation column which produces an overhead stream comprising butyl acetate and n-butanol, and a bottom stream rich in n-butyl acrylate;
n) feeding a portion of the overhead stream comprising butyl acetate and n-butanol from step m) to the neutralization and acidification system, and to the esterification reactor recycle tank, from which a recycle feed stream is sent to the esterification reactor.

### BRIEF DESCRIPTION OF THE DRAWINGS

FIG. 1 is a schematic depiction of equipment and flow lines for two process units being run together to produce n-butyl acrylate.

FIG. 2 is a schematic depiction of equipment and flow lines for a process unit using both a neutralization and acidification system and a hydrolysis reactor.

### DETAILED DESCRIPTION OF THE INVENTION

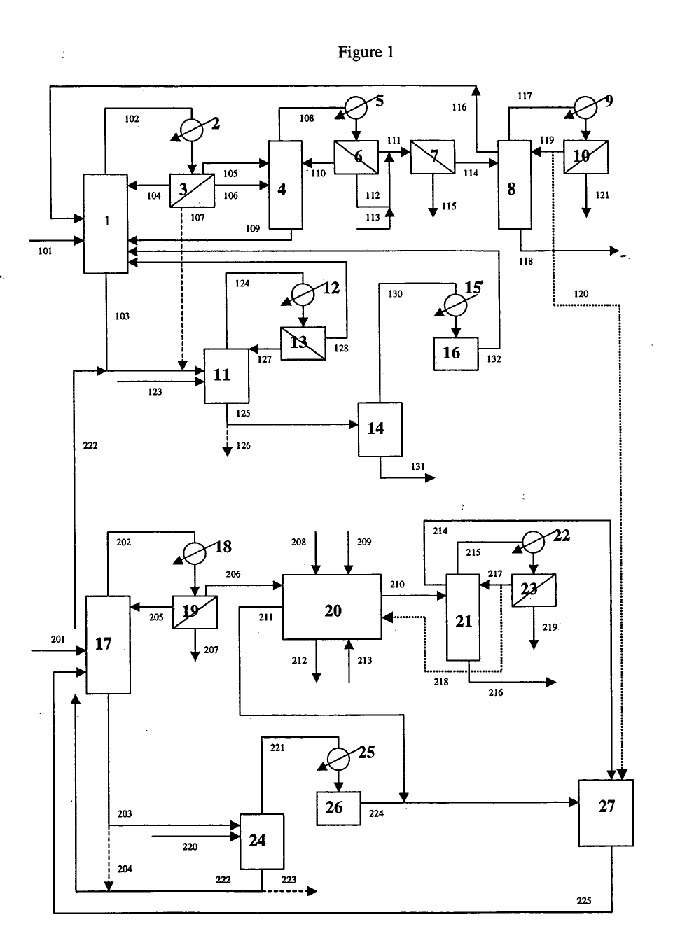

In operation of the two process units depicted in FIG. 1, fresh acrylic acid, n-butanol, and esterification catalyst are fed to the first unit esterification reactor 1 via line 101. Suitable fresh acrylic acid may include crude acrylic acid, commercially-available grades of acrylic acid, such as, for example, Glacial Acrylic Acid (GAA) or Floculant Grade Acrylic Acid (FGAA), and mixtures thereof. Fresh acrylic acid may comprise one or more components selected from the list including polymerization inhibitors, water, acrylic acid dimer, acetic acid, or propionic acid. Commercially available n-butanol is suitable for the process of the present invention. Suitable esterification catalysts include, for example: sulfuric acid and strong acid ion-exchange resins; esterification catalysts may be used at concentrations ranging from 1% to 10% by weight as measured in the esterification reactor bottoms. The esterification reactor temperature is maintained at from 74 to 105° C., at reactor pressures from 60-200 mm Hg. At least one heat exchanger may be used to control the temperature of esterification reactor 1. Desuperheated steam is preferred as the heat exchanger's heat source. In one embodiment, an external reboiler (not shown) is used as the heat exchanger for esterification reactor 1, and at least a portion of the reactor contents are passed through the reboiler via a reboiler recirculation line (not shown). Oxygen-containing gas, such as for example air, may be optionally admixed into the reboiler circulation line to ensure polymerization inhibitor efficacy within the esterification reactor 1 and its reboiler. Esterification reactor vaporized mixture is distilled overhead via line 102 to condenser 2, and reactor bleed is sent to the hydrolysis reactor unit (HRU) 11 via line 103. Condensed reactor overhead is sent to separator 3, and controlled portions of the organic and aqueous layers are sent to the acrylic acid separation column 4 via lines 105 and 106, while a controlled portion of the organic layer is refluxed to the entrainment separator (not shown) surmounting reactor 1 via line 104. A portion of the aqueous layer may be sent to the HRU 11 via line 107. A portion of the aqueous layer may also be sent to the alcohol stripper (not shown), while other additional aqueous streams that do not contain sulfates are sent to the acrylic acid separation column 4 to minimize potential corrosion.

Lines from the acrylic acid separation column 4 include 109, returning the AA-rich bottoms stream to reactor 1, and line 108 conveying the distilled overhead mixture through condenser 5 to phase separator 6, and its associated lines, line 110 returning a controlled portion of the aqueous phase to the top of the acrylic acid separation column 4, line 112 moving forward a controlled portion of the separated aqueous phase, and line 111 carrying forward all of the BA-rich organic phase to separator 7. Line 113 provides dilute caustic to neutralize any residual AA in the acrylic acid column overhead feeding separator 7.

The upper layer of separator 7 is fed to the dehydration column 8, while the aqueous layer is sent to an aqueous recovery column (alcohol stripper—not shown) to recover organics and minimize wastewater from the process. Excess n-butanol, water, and light-end byproducts are distilled overhead in the dehydration distillation column 8. A liquid side-draw stream is sent back to reactor 1 for recycle via line 116, while the remaining overhead vapor is sent to condenser 9 via line 117. Optionally, streams 109 and 116 are sent to a first-unit recycle tank where they are combined, and then sent to reactor 1. Optionally, a portion of the side-draw stream 116 may be sent to the hydrolysis reactor separator 13 to enhance separation. Dehydration column 8 bottoms product BA is sent forward for final processing via line 118. In one embodiment, such final processing is accomplished in one or more final product BA distillation columns (not shown) to provide final product butyl acrylate meeting commercial quality specifications, and a bottoms stream comprising residual BA, inhibitors, and impurities. As with all polymerizable monomers, it is beneficial for the final product butyl acrylate to comprise polymerization inhibitors in order to prevent polymerization in shipment and storage. MeHQ is most commonly utilized as a final product BA inhibitor, and is typically maintained at a concentration of between 10 ppm and 20 ppm in the final product BA. Inhibitor solutions comprising MeHQ may be provided to the final product distillation columns to achieve this final product inhibitor concentration. Oxygen-containing gas, such as for example air, may also be added to the final product distillation columns. In some embodiments, a variable amount MeHQ inhibitor may be added directly to the butyl acrylate product stream enroute to final product storage, thereby ensuring that the final product BA stream's inhibitor concentration remains within specification. The overhead material from dehydration column 8 is condensed and sent to separator 10, where a portion of the organic layer is refluxed to the dehydration column 8 via line 119. The water layer from separator 10 is sent to the alcohol stripper via line 121. Optionally, the water layer from the separator 10 may be sent to the acrylic acid separation column 4 as part of the aqueous feeds. An organic bleed stream containing n-butanol and light-end byproducts is sent to the second unit recycle feed tank 27 via line 120 to improve first unit product purity. This bleed stream is combined with all other second unit recycle feeds and analyzed on-line to improve second unit reactor feed control consistency. The stream transferred via line 120 represents 1 to 15% of the overhead stream, preferably 1 to 10%, more preferably 2 to 7%, and most preferably about 3%.

First unit reactor 1 bleed is fed to the hydrolysis reactor unit (HRU) 11 via line 103, along with optional SBA bleed stripper 24 bottoms via line 222, and other optional bottoms streams containing acrylic acid via line 123. Mineral acid, for example sulfuric acid or a sulfonic acid, may optionally be added to HRU 11 as well. HRU 11 overhead is sent to condenser 12 via line 124, while bottoms are sent to the first unit cracking reactor 14 via line 125. HRU bottoms may be sent directly to an acid recovery unit (not shown) via line 126, but AA yields will be lower. Condensed HRU overhead is sent to separator 13, where the organic phase is sent back to reactor 1 via line 128, and the aqueous layer is sent back to the HRU 11 via line 127. Overhead from the cracking reactor 14 is sent to condenser 15 via line 130, and condensed distillate is sent to receiver 16 and then recycled back to reactor 1 via line 132. The bottoms residue from the cracking reactor 14 is sent to an acid recovery unit (not shown) via line 131. Optionally, streams 128 and 132 are sent to a first-unit recycle tank (not shown) where they are combined, and then sent to reactor 1.

In the second unit, fresh acrylic acid, n-butanol, and esterification catalyst are fed to the second unit esterification reactor 17 via line 201. Suitable fresh acrylic acid may include crude acrylic acid, available-available grades of acrylic acid, such as, for example, Glacial Acrylic Acid (GAA) or Floculant Grade Acrylic Acid (FGAA), and mixtures thereof. Fresh acrylic acid may comprise one or more components selected from the list including polymerization inhibitors, water, acrylic acid dimer, acetic acid, or propionic acid. Commercially available n-butanol is suitable for the process of the present invention. Suitable esterification catalysts include, for example: sulfuric acid and strong acid ion-exchange resins; esterification catalysts may be used at concentrations ranging from 1% to 10% by weight as measured in the esterification reactor bottoms. The esterification reactor temperature is maintained at from 74 to 105° C., at reactor pressures from 60-200 mm Hg. At least one heat exchanger may be used to control the temperature of esterification reactor 17. Desuperheated steam is preferred as the heat exchanger's heat source. In one embodiment, an external reboiler (not shown) is used as the heat exchanger for esterification reactor 17, and at least a portion of the reactor contents are passed through the reboiler via a reboiler recirculation line (not shown). Oxygen-containing gas, such as for example air, may be optionally admixed into the reboiler circulation line to ensure polymerization inhibitor efficacy within the esterification reactor 17 and its reboiler. Esterification reactor vaporized mixture is distilled overhead via line 202 to condenser 18, and reactor bleed is sent to the bleed stripper 24 via line 203. Reactor bottoms may bypass the bleed stripper 24 and be sent to the first unit HRU 11 via line 204. Condensed reactor overhead is sent to separator 19, and a controlled portion of the organic layers is sent forward to the neutralization/acidification system 20 via 206, while a controlled portion of the organic layer is refluxed to the entrainment separator surmounting reactor 17 via line 205. The aqueous layer is sent to an aqueous recovery column (alcohol stripper—not shown) via line 207.

Esterification organic layer from separator 19 is neutralized with a base, for example, caustic fed via line 208 to the neutralization/acidification system 20. The neutralization/acidification system 20 may comprise one or more of mixers, separators, extraction units, centrifugal separators, etc., known in the art and suitable for neutralizing and separating acrylic acid from acrylate esters and alcohols. Aqueous sodium acrylate is re-acidified with sulfuric acid fed to the system via line 209. An aqueous stream containing sodium sulfate is sent to the alcohol stripper (not shown) via line 212 for n-butanol recovery. A recovered AA organic stream is sent to the recycle feed tank 27 via line 211. Additional aqueous and organic streams are sent to the neutralization/acidification system 20 to enhance AA recovery: from the alcohol stripper via line 213, and from the dehydration column separator 23 organic layer via line 218. Hydrolysis of some light-ends byproducts, e.g., n-butyl acetate, occurs in the neutralization/acidification system.

Crude BA is fed to the dehydration distillation column 21 from the neutralization/acidification system via line 210. Excess n-butanol, water, and light-end byproducts are distilled overhead in the dehydration distillation column 21. A liquid side-draw stream is sent to the recycle feed tank 27 via line 214, while the remaining overhead vapor is sent to condenser 22 via line 215. Dehydration distillation column 21 bottoms product BA is sent forward for final processing via line 216. In one embodiment, such final processing is accomplished in one or more final product BA distillation columns (not shown) to provide final product butyl acrylate meeting commercial quality specifications, and a bottoms stream comprising residual BA, inhibitors, and impurities. In one embodiment, dehydration column 21 bottoms product BA is combined with dehydration column 8 bottoms product BA, and final processing is performed in a common system comprising one or more final product BA distillation columns (not shown). The overhead material from dehydration column 21 is condensed and sent to separator 23, where a portion of the organic layer is refluxed to the dehydration distillation column 21 via line 217. The water layer from separator 23 is sent to the alcohol stripper (not shown) via line 219. An organic bleed stream containing n-butanol and light-end byproducts is sent to the neutralization/acidification system 20 via line 218 to act as an extraction solvent to recover AA from aqueous sodium sulfate.

Second unit reactor 17 bleed is fed to the second unit bleed stripper 24 via line 203, along with optional bottoms streams containing acrylic acid via line 220. Bleed stripper distillate 24 is sent to condenser 25 via line 221, and condensed distillate is sent to receiver 26 and then to recycle feed tank 27 via line 224. The bottoms residue from the second unit bleed stripper 24 is sent to the first unit HRU 11 for further recovery via line 222. Residue may be sent directly from the bleed stripper 24 to an acid recovery unit via line 223, but acrylic acid and n-butanol yields will be lower.

Combined second unit recycle streams from recycle feed tank 27 are fed back to the second unit reactor 17 via line 225. The material may be analyzed on-line for improved reactor feed ratio control.

When the two process units were run as described in FIG. 1 for a period of several days, n-butyl acrylate product was produced having less than 500 ppm n-butyl acetate and less than 0.009% of acrylic acid. When the first process unit is run by itself, butyl acetate levels typically exceed 500 ppm.

The first and second process units depicted in FIG. 1 comprise the following major vessels. The first process unit includes esterification reactor 1, acrylic acid separation column 4, dehydration distillation column 8, hydrolysis reactor 11 and cracking reactor 14. The second process unit includes esterification reactor 17, neutralization/acidification system 20, dehydration distillation column 21, bleed stripper 24 and recycle tank 27.

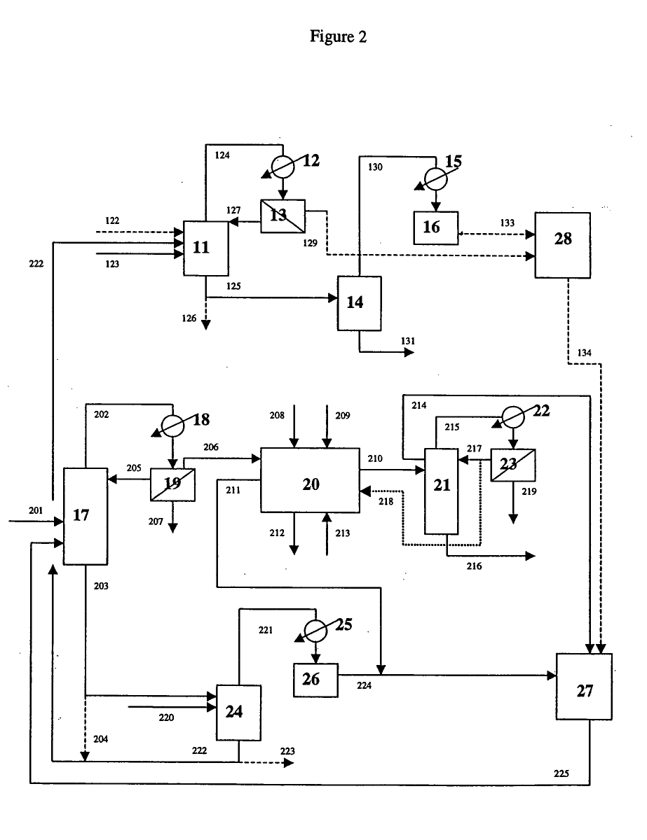

In the process unit configuration depicted in FIG. 2, fresh acrylic acid, n-butanol, and esterification catalyst are fed to the esterification reactor 17 via line 201. Suitable fresh acrylic acid may include crude acrylic acid, commercially-available grades of acrylic acid, such as, for example, Glacial Acrylic Acid (GAA) or Floculant Grade Acrylic Acid (FGAA), and mixtures thereof. Fresh acrylic acid may comprise one or more components selected from the list including polymerization inhibitors, water, acrylic acid dimer, acetic acid, or propionic acid. Commercially available n-butanol is suitable for the process of the present invention. Suitable esterification catalysts include, for example: sulfuric acid and strong acid ion-exchange resins; esterification catalysts may be used at concentrations ranging from 1% to 10% by weight as measured in the esterification reactor bottoms. The esterification reactor temperature is maintained at from 74 to 105° C., at reactor pressures from 60-200 mm Hg. Esterification reactor vaporized mixture is distilled overhead via line 202 to condenser 18, and reactor bleed is sent to the bleed stripper 24 via line 203. Reactor bottoms may bypass the bleed stripper 24 and be sent to the HRU 11 via line 204. Condensed reactor overhead is sent to separator 19, and a controlled portion of the organic layers is sent forward to the neutralization/acidification system 20 via 206, while a controlled portion of the organic layer is refluxed to the entrainment separator (not shown) surmounting reactor 17 via line 205. The aqueous layer is sent to an aqueous recovery column (alcohol stripper—not shown) via line 207.

Esterification organic layer from separator 19 is neutralized with a base, for example, caustic fed via line 208 to the neutralization/acidification system 20. The neutralization/acidification system 20 may comprise one or more of mixers, separators, extraction units, centrifugal separators, etc., known in the art and suitable for neutralizing and separating acrylic acid from acrylate esters and alcohols. Aqueous sodium acrylate is re-acidified with sulfuric acid fed to the system via line 209. An aqueous stream containing sodium sulfate is sent to the alcohol stripper (not shown) via line 212 for n-butanol recovery. A recovered AA organic stream is sent to the recycle feed tank 27 via line 211. Additional aqueous and organic streams are sent to the neutralization/acidification system 20 to enhance AA recovery: from the alcohol stripper via line 213, and from the dehydration distillation column separator 23 organic layer via line 218. Hydrolysis of some light-ends byproducts, e.g., n-butyl acetate, occurs in the neutralization/acidification system.

Crude BA is fed to the dehydration distillation column 21 from the neutralization/acidification system via line 210. Excess n-butanol, water, and light-end byproducts are distilled overhead in the dehydration distillation column 21. A liquid side-draw stream is sent to the recycle feed tank 27 via line 214, while the remaining overhead vapor is sent to condenser 22 via line 215. Dehydration distillation column 21 bottoms product BA is sent forward for final processing via line 216. Condensed overhead material is sent to separator 22, where a portion of the organic layer is refluxed to the dehydration distillation column 21 via line 217. The water layer from separator 23 is sent to the alcohol stripper (not shown) via line 219. An organic bleed stream containing n-butanol and light-end byproducts is sent to the neutralization/acidification system 20 via line 218 to act as an extraction solvent to recover AA from aqueous sodium sulfate.

Reactor 17 bleed is fed to the bleed stripper 24 via line 203, along with optional bottoms streams containing acrylic acid via line 220. Bleed stripper 24 distillate is sent to condenser 25 via line 221, and condensed distillate is sent to receiver 26 and then to recycle feed tank 27 via line 224. The bottoms residue from the bleed stripper 24 is sent to the HRU 11 for further recovery via line 222. Residue may be sent directly from the bleed stripper 24 to an acid recovery unit via line 223, but acrylic acid and n-butanol yields will be lower.

Water is fed to the hydrolysis reactor unit (HRU) 11 via line 122, along with SBA bleed stripper 24 bottoms via line 222, and other optional bottoms streams containing acrylic acid via line 123. HRU 11 overhead is sent to condenser 12 via line 124, while bottoms are sent to the cracking reactor 14 via line 125. HRU bottoms may be sent directly to an acid recovery unit (not shown) via line 126, but acrylic acid yields will be lower. Condensed HRU overhead is sent to separator 13, where the organic phase is sent to the AA/Slops tank 28 via line 129, and the aqueous layer is sent back to the HRU 11 via line 127. Overhead from the cracking reactor 14 is sent to condenser 15 via line 130, and condensed distillate is sent to receiver 16 and then to the AA/Slops tank 28 via line 133. The bottoms residue from the cracking reactor 14 is sent to an acid recovery unit (not shown) via line 131. The streams sent to tank 28 may alternatively be sent directly to recycle feed tank 27.

Material in the AA/Slops tank 28 is sent to the recycle feed tank 27. Combined recycle streams from recycle feed tank 27 are fed back to the reactor 17 via line 225. The material may be analyzed on-line for improved reactor feed ratio control.

When the configuration of FIG. 2 was run for several days, n-butyl acrylate product was produced with at least 2% higher yields on acrylic acid and n-butanol compared to a similar system run without the hydrolysis recovery unit or the cracking reactor.

The hydrolytic recovery method of any embodiment of the invention may be carried out in a multi-plate reactive distillation column or other staged reactor, and preferably is carried out under continuously mixed conditions, as in a continuous flow stirred tank reactor (“CSTR”). By “bleed stream” is meant any process stream which is controllably withdrawn from one vessel to another, such as from a reactor to another reactor or distillation column. Sulfuric acid is most preferred for use as both reactor acid catalyst and mineral acid in all embodiments of the invention. The HRU mixture of the described feed streams is maintained in a boiling state under the conditions defined. The residence time of from 0.5 to 20 hours is based on the total aqueous and organic feed stream (“total” meaning the sum of the aqueous and heavy ends and/or reactor bleed streams) fed to the HRU. Preferred residence time is from 0.5 to 5 hrs, and more preferred is 0.5 to 3 hrs. The cracking reactor may be of construction similar to that of the HRU and is, preferably, a CSTR. The cracking reactor liquid is maintained at least at 7.5 wt. % mineral acid, preferably sulfuric, and also contains a mixture of acrylic acid, n-butanol, BA, some heavy ends and residual polymerization inhibitors. Additional mineral or sulfonic acid may be added to the cracking reactor liquid (feed line not shown).

The HRU may be a multi-plate reactive distillation column so long as sufficient number of plates are incorporated to provide specified residence time. When a reactive distillation column is employed as an HRU, a separate cracking reactor unit may not be needed to achieve acceptable values recoveries. Under most production conditions it is preferred to use the cracking reactor in tandem with a hydrolytic reactive distillation column, similar to its use when the HRU is a CSTR. One disadvantage of a reactive distillation column over a CSTR is that occasional build-up of solids on the column trays may require undesirable down time for column cleaning.

Addition of one or more additional feed streams to the esterification reactor bleed stream or directly to the hydrolysis reactor permits additional recovery of AA, and for example, BA, and n-butanol, through the processes occurring in the hydrolysis reactor and, when used, the cracking reactor. The liquid in the hydrolysis reactor has at least 5 wt. % water for efficient operation; preferably the HRU liquid contains from 9 to 18 wt. %, more preferably from 10 to 16 wt. % water, in order to achieve efficient hydrolysis rates under nominal thermal and pressure conditions and practical equipment size. Water content is maintained by a combination of returning the entire condensed and separated aqueous stream to the hydrolysis reactor and by adding additional water from other lines to compensate for water losses to organic distillate and the HRU bleed stream. Water addition from the distilled aqueous phase from the esterification reactor is a preferred source of water in the continuous BA process. In order to maintain efficient dehydration and retro-Michael reaction rates in the cracking reactor, the cracking reaction mixture should have an aqueous content lower than that of the HRU mixture. Water contents typically below 5 wt. %, preferably below 1 wt. %, are achieved by operating the cracking reactor as a single stage unit, that is, by continuously distilling from the cracking reactor any water carried over from the hydrolysis reactor bleed stream and any additional water generated from cracking reactions.

Additional acid may be added to the recovery units as necessary to achieve practical reaction rates; preferably acid is added by way of one or more of the feed streams. “Residual acid catalyst” is acid catalyst which remains present as acid in the esterification reactor bleed stream and thus is carried forward to the HRU. In the HRU, acid concentration is preferably in the range of 3.5 to 15 wt. %, and most preferably is 5 to 8 wt. %. Acid concentration in the cracking reactor is typically in the range of 7.5 to 20 wt. %, and could be higher, e.g. up to 50%. Acid concentration preferably is from 10 to 13 wt. %, particularly for BA production. The amount of heavy ends in the esterification reactor bleed stream may vary but typically is in the range of from 10 to 50 wt. % of the combined total of the aqueous and organic-containing feed stream.

Hydrolysis reaction temperatures range from 90° to 140° C., and are preferably from 105° to 125° C. for efficient hydrolysis rates; temperatures greater than 140° C. may lead to thermally induced polymerization of alkyl acrylates and of acryloxy-bearing heavy ends, resulting in undesired product loss. The residence time required for HRU hydrolysis reaction is preferably from 0.5 to 5 hours, more preferably from 0.5 to 3 hours, shorter times being more economical. Lower temperatures, and the presence of water, also favor reduced DBE formation. Cracking reactor temperatures range from 90° to 140° C., preferably from 110 to 125° C.; cracking pressures typically range from 20 mm Hg to 200 mm Hg, although higher pressures, up to 800 mm Hg may be used. The residence time for dehydration and other reactions in the cracking reactor under these conditions is preferably from 0.5 to 3 hours. For the continuous production of BA, values recoveries are maximized with two CSTR reactors in tandem, one the HRU and the other the cracking reactor.

In order to prevent polymerization, an effective amount of one or more polymerization inhibitor may be added at any step in any component of the process. An esterification reactor process stream typically contains sufficient inhibitor to prevent polymerization in the HRU and cracking reactor. If additional inhibitor is required, any of a large number of known inhibitors may be used, for example, hydroquinone (HQ), 4-methoxyphenol (MEHQ), 4-ethoxyphenol, 4-propoxyphenol, 4-butoxyphenol, 4-heptoxyphenol, hydroquinone monobenzylether, 1,2-dihydroxybenzene, 2-methoxyphenol, 2,5-dichlorohydroquinone, 2,5-di-tert-butylhydroquinone, 2-acetylhydroquinone, hydroquinone monobenzoate, 1,4-dimercaptobenzene, 1,2-dimercaptobenzene, 2,3,5-trimethylhydroquinone, 4-aminophenol, 2-aminophenol, 2-N,N-dimethylaminophenol, 2-mercaptophenol, 4-mercaptophenol, catechol monobutylether, 4-ethylaminophenol, 2,3-dihydroxyacetophenone, pyrogallol-1,2-dimethylether, 2-methylthiophenol, t-butyl catechol, di-tert-butylnitroxide, di-tert-amyInitroxide, 2,2,6,6-tetramethyl-piperidinyloxy, 4-hydroxy-2,2,6,6-tetramethyl-piperidinyloxy, 4-oxo-2,2,6,6-tetramethyl-piperidinyloxy, 4-dimethylamino-2,2,6,6-tetramethyl-piperidinyloxy, 4-amino-2,2,6,6-tetramethyl-piperidinyloxy, 4-ethanoloxy-2,2,6,6-tetramethyl-piperidinyloxy, 2,2,5,5-tetramethyl-pyrrolidinyloxy, 3-amino-2,2,5,5-tetramethyl-pyrrolidinyloxy, 2,2,5,5-tetramethyl-1-oxa-3-azacyclopentyl-3-oxy, 2,2, 5,5-tetramethyl-3-pyrrolinyl-1-oxy-3-carboxylic acid, 2,2,3,3,5,5,6,6-octamethyl-1,4-diazacyclohexyl-1,4-dioxy, salts of 4-nitrosophenolate, 2-nitrosophenol, 4-nitrosophenol, copper dimethyldithiocarbamate, copper diethyldithiocarbamate, copper dibutyldithiocarbamate, copper salicylate, methylene blue, iron, phenothiazine (PTZ), 3-oxophenothiazine, 5-oxophenothiazine, phenothiazine dimer, 1,4-benzenediamine, N-(1,4-dimethylpentyl)-N′-phenyl-1,4-benzenediamine, N-(1,3-dimethylbutyl)-N′-phenyl-1,4-benzenediamine, N-nitrosophenyl hydroxylamine and salts thereof, nitric oxide, nitrosobenzene, p-benzoquinone, copper naphthenate, copper acetate, manganese dimethyl dithiocarbamate, manganese diethyldithiocarbamate, manganese dibutyldithiocarbamate, manganese naphthenate, manganese acetate, manganese acetylacetonate, cobalt acetate, cobalt carbonate, cobalt acetate, nitrogen dioxide, nitrobenzene, nitrosobutane, N-nitrosodiphenylamine, diphenylphenylenediamine, nitrosocarbazole, 1-nitroso-2-naphthol, 2,4 dinitrobenzene, triphenyl phosphine, triethyl phosphine, tributyl phosphine, triphenyl phosphite, triethyl phosphite, tri-iso-propylphosphite, tributyl phosphite, tricyclohexyl phosphite, sodium bisulfite, butyl mercaptan, dodecyl mercaptan, N,N-diethylhydroxylamine, or isomers thereof, mixtures of two or more thereof, mixtures of one or more of the above with molecular oxygen. The inhibitor(s) may be used alone or combined with a suitable diluent. The polymerization inhibitor is typically used at levels ranging from 100 ppm to 4,000 ppm by weight. Separations column bottoms comprising inhibitors may be recycled to other parts of the process for cost savings. PTZ may optionally be added to the recycle inhibitor. In addition to the use of polymerization inhibitors, separation columns and other process equipment may optionally be operated under a vacuum (i.e., below atmospheric pressure) to minimize monomer processing temperatures, thereby limiting thermally-induced polymerization. Additionally, desuperheated steam may be used as the heating medium in reboilers, such as for example distillation column reboilers, to further limit polymer formation.

Because the stripper may have sieve trays, a vapor phase inhibitor such as n-phenyl hydroxylamine or derivatives thereof may be useful. Liquid phase inhibitors may also be useful. In a preferred embodiment, the vapor phase inhibitor is added to the reboiler and the bottom trays of the column, while the liquid phase inhibitor is added to the top of the column. The amount of liquid phase inhibitor may range from 1 ppm to 1000 ppm, depending on the feed rate to the column.

Distillation of crude BA in the acrylic acid separation column provides BA substantially free of AA by efficiently handling distillate and aqueous reflux. Specifically, the column provides BA in the BA-rich stream containing less than 2,000 ppm of AA for use in subsequent conventional isolation. The method also provides an AA recycle stream containing negligible BA, specifically providing an AA recycle stream (the bottom, AA-rich phase) containing less than 10 ppm, preferably less than 5 ppm, of BA. In generating the crude BA for the dehydration distillation column, AA and n-butanol are initially fed, along with acid catalyst, to an esterification reactor in a molar ratio of AA to n-butanol in the range of 1:1.1 to 1:1.7, preferably 1:1.25 to 1:1.45, and reacted to a conversion on AA of from 60 to 95%, preferably 75 to 85%, using an acid catalyst of the mineral or sulfonic acid type previously described, or a strong acid ion exchange resin; preferably sulfuric acid is used. The reactant ratio and BA conversion provide a crude BA stream which may be processed to provide stable “aqueous mode” operation of the acrylic acid separation column. Reactor contents are maintained in a boiling state during continuous distillation of the vaporized mixture of AA, BA, n-butanol and water.

1. A continuous process for producing n-butyl acrylate substantially free of acrylic acid and n-butyl acetate and for recovering acrylic acid, n-butyl acrylate, n-butanol and water from an esterification reactor mixture containing acrylic acid, n-butyl acrylate, n-butanol, water, heavy ends, and acid catalyst; wherein two process units are run in parallel, each comprising an esterification reactor and a dehydration distillation column; and wherein a first process unit has a hydrolysis reactor, a cracking reactor and an acrylic acid separation column and a second process unit has a bleed stripper, a recycle tank and a neutralization and acidification system; comprising steps of, with each step occurring in both the first and the second process unit unless otherwise indicated:
a) feeding to the esterification reactors acrylic acid and n-butanol, in a molar ratio of from 1 to 1.1 to 1 to 1.7, and the acid catalyst;
b) reacting the acrylic acid and n-butanol to yield n-butyl acrylate in a conversion of at least 60% on acrylic acid, and yielding the esterification reaction mixture comprising acrylic acid, n-butyl acrylate, n-butanol, di-n-butyl ether, n-butyl acetate, water, heavy ends, and acid catalyst;
c) withdrawing a reactor bleed stream from the continuously converting esterification reactor mixture while concurrently distilling acrylic acid, n-butyl acrylate, n-butanol and water from the esterification reaction mixture;
d) feeding the reactor bleed stream from the second process unit reactor to a bleed stripper unit from which a distillate comprising acrylic acid, n-butyl acrylate and n-butanol is removed;
e) feeding total aqueous and organic feed streams from each process unit comprising the reactor bleed stream from the first process unit and the bleed stripper bottom stream from the second process unit, water or reactor aqueous distillate from the first process unit, optionally a strong acid selected from a mineral acid or sulfonic acid, and optionally additional heavy ends, to the hydrolysis reactor in the first process unit, which is maintained at 90° to 140° C., 50 to 1000 mm Hg pressure, and a residence time of 0.5 to 20 hours based on the total aqueous and organic feed stream;
f) in the first process unit, distilling an overhead stream containing acrylic acid, n-butyl acrylate, n-butanol, and water from the hydrolysis reactor while maintaining a hydrolysis reactor liquid concentration of from 5 to 40 weight % water and at least 1 weight % acid, the acid comprising the acid catalyst and the optional strong acid; and condensing the overhead stream;
g) in the first process unit, separating from the condensed overhead stream from step l) an organic phase comprising n-butyl acrylate, n-butanol, and acrylic acid, and an aqueous phase comprising primarily water, and acrylic acid, and n-butanol; feeding the separated organic phase to the esterification reactor; and feeding the separated aqueous phase to the hydrolysis reactor;
h) in the first process unit, withdrawing from the hydrolysis reactor from 20 to 70 weight %, based on the total aqueous and organic feed stream, of a hydrolysis reactor bleed stream; and feeding up to 100% of the hydrolysis reactor bleed stream to a cracking reactor maintained at 90° to 140° C., a pressure of from 20 to 200 mm Hg, and a residence time of 0.5 to 20 hours based on the fed reactor bleed stream;
i) in the first process unit, distilling from the cracking reactor a cracking reactor overhead stream comprising acrylic acid, n-butyl acrylate, n-butanol, and water while maintaining a cracking reactor liquid concentration of at least 7.5 weight % acid; condensing the cracking reactor overhead stream; and recycling to the esterification reactor the condensed cracking reactor overhead stream comprising acrylic acid, n-butyl acrylate, n-butanol, and water;
j) distilling from the esterification reactor, concurrently with above steps c) through i), a vaporized mixture comprising acrylic acid, n-butyl acrylate, n-butanol, and water;
k) condensing the vaporized mixture to provide a first condensate comprising an organic phase and an aqueous phase; returning from 0 to 30 percent of the organic phase to an entrainment separator surmounting the esterification reactor;
l) in the first process unit, feeding from 70 to 100 percent of the organic phase and from 50 to 100 percent of the aqueous phase to an acrylic acid separation column; distilling from the acrylic acid separation column, at a pressure of from 35 to 800 mm Hg, in an aqueous mode and at an aqueous reflux ratio of 8.5:1 to 17:1, an overhead mixture comprising an azeotropic mixture of n-butanol, butyl acrylate and water; and removing from the distillation column an acrylic acid-rich bottom stream;
m) in the first process unit, recycling the acrylic acid-rich bottom stream from the acrylic acid separation column to the esterification reactor; condensing the overhead mixture to provide a second condensate; separating the second condensate into a n-butyl acrylate-rich organic phase and a separated aqueous phase; and removing the n-butyl acrylate-rich organic phase substantially free of acrylic acid;
n) in the second process unit, feeding from 70 to 100 percent of the organic phase from step k) to the neutralization and acidification system to produce an n-butyl acrylate-rich organic phase low in acrylic acid;
o) feeding an n-butyl acrylate-rich organic phase low in acrylic acid from step m) in the first process unit or step n) in the second process unit to the dehydration distillation columns, which produce overhead streams comprising butyl acetate, n-butanol and n-butyl acrylate, and bottom streams rich in n-butyl acrylate;
p) feeding a portion from 1 to 15% of the overhead stream comprising butyl acetate, n-butanol and n-butyl acrylate from step o) in the first process unit to the recycle tank in the second process unit, from which a recycle feed stream is sent to the second process unit esterification reactor.

2. A continuous process for producing n-butyl acrylate substantially free of acrylic acid and n-butyl acetate and for recovering acrylic acid, n-butyl acrylate, n-butanol and water from an esterification reactor mixture containing acrylic acid, n-butyl acrylate, n-butanol, water, heavy ends, and acid catalyst; comprising steps of
a) feeding to an esterification reactor acrylic acid and n-butanol, in a molar ratio of from 1 to 1.1 to 1 to 1.7, and the acid catalyst;
b) reacting the acrylic acid and n-butanol to yield n-butyl acrylate in a conversion of at least 60% on acrylic acid, and yielding the esterification reaction mixture comprising acrylic acid, n-butyl acrylate, n-butanol, di-n-butyl ether, n-butyl acetate, water, heavy ends, and acid catalyst;
c) withdrawing a reactor bleed stream from the continuously converting esterification reactor mixture while concurrently distilling acrylic acid, n-butyl acrylate, n-butanol and water from the esterification reaction mixture;
d) feeding the reactor bleed stream to a bleed stripper unit from which a distillate comprising acrylic acid and n-butyl acrylate is removed;
e) feeding the bleed stripper bottom stream, water, optionally a strong acid selected from a mineral acid or sulfonic acid, and optionally additional heavy ends, to a hydrolysis reactor, which is maintained at 90° to 140° C., 50 to 1000 mm Hg pressure, and a residence time of 0.5 to 20 hours based on the total aqueous and organic feed stream;
f) distilling an overhead stream containing acrylic acid, n-butyl acrylate, n-butanol, and water from the hydrolysis reactor while maintaining a hydrolysis reactor liquid concentration of from 5 to 40 weight % water and at least 1 weight % acid, the acid comprising the acid catalyst and the optional strong acid; and condensing the overhead stream;
g) separating from the condensed overhead stream from step 1) an organic phase comprising n-butyl acrylate, n-butanol, and acrylic acid, and an aqueous phase comprising primarily water, and acrylic acid, and n-butanol; feeding the separated organic phase to an esterification reactor recycle tank; and feeding the separated aqueous phase to the hydrolysis reactor;
h) withdrawing from the hydrolysis reactor from 20 to 70 weight %, based on the total aqueous and organic feed stream, of a hydrolysis reactor bleed stream; and feeding up to 100% of the hydrolysis reactor bleed stream to a cracking reactor maintained at 90° to 140° C., a pressure of from 20 to 200 mm Hg, and a residence time of 0.5 to 20 hours based on the fed reactor bleed stream;
i) distilling from the cracking reactor a cracking reactor overhead stream comprising acrylic acid, n-butyl acrylate, n-butanol, and water while maintaining a cracking reactor liquid concentration of at least 7.5 weight % acid; condensing the cracking reactor overhead stream; and recycling to the esterification reactor recycle tank the condensed cracking reactor overhead stream comprising acrylic acid, n-butyl acrylate, n-butanol, and water;
j) distilling from the esterification reactor, concurrently with above steps c) through i), a vaporized mixture comprising acrylic acid, n-butyl acrylate, n-butanol, and water;
k) condensing the vaporized mixture to provide a first condensate comprising an organic phase and an aqueous phase; returning from 0 to 30 percent of the organic phase to an entrainment separator surmounting the esterification reactor;
l) feeding from 70 to 100 percent of the organic phase from step k) to the neutralization and acidification system to produce an n-butyl acrylate-rich organic phase low in acrylic acid;
m) feeding an n-butyl acrylate-rich organic phase low in acrylic acid from step l) to a dehydration distillation column which produces an overhead stream comprising butyl acetate and n-butanol, and a bottom stream rich in n-butyl acrylate;
n) feeding a portion of the overhead stream comprising butyl acetate and n-butanol from step m) to the neutralization and acidification system, and to the esterification reactor recycle tank, from which a recycle feed stream is sent to the esterification reactor.
